(19) United States
(12) Patent Application Publication
(10) Pub. No.: US 2012/0322065 A1
(43) Pub. Date: Dec. 20, 2012

(54) Methods for Use with Nanoreactors

(76) Inventors: Michael S. Urdea, Emeryville, CA (US); Theodore M. Tarasow, Emeryville, CA (US); Edward J. Moler, Emeryville, CA (US)

(21) Appl. No.: 13/477,961

(22) Filed: May 22, 2012

Related U.S. Application Data
(63) Continuation of application No. 12/436,585, filed on May 6, 2009, now abandoned.
(60) Provisional application No. 61/050,932, filed on May 6, 2008.

(51) Int. Cl. G01N 33/558; G01N 33/566; G01N 27/62; G01N 21/65; G01N 21/25; C12Q 1/68; G01N 21/64

(52) U.S. Cl. 435/6.11; 435/7.9; 435/7.94; 436/501

(57) ABSTRACT

The invention relates to methods of using nanoreactor technology for sample analysis in microfluidic systems.

### CROSS-REFERENCE TO RELATED APPLICATIONS

This application claims the priority benefit of U.S. Provisional Application Ser. No. 61/050,932, filed May 6, 2008, which is hereby incorporated by reference herein in its entirety.

### FIELD OF THE INVENTION

The present invention relates generally to methods for using nanoreactor technology for sample analysis in microfluidic systems.

### BACKGROUND OF THE INVENTION

Microfluidic technology has been applied to high throughput screening methods. For example, U.S. Pat. Nos. 6,508,988; and 5,942,056.

The manipulation of fluids to form fluid streams of desired configuration, discontinuous fluid streams, droplets, particles, dispersions, etc., for purposes of fluid delivery, product manufacture, analysis, and the like have been described. For example, highly monodisperse gas bubbles, less than 100 microns in diameter, have been produced using a technique referred to as capillary flow focusing. In this technique, gas is forced out of a capillary tube into a bath of liquid, the tube is positioned above a small orifice, and the contraction flow of the external liquid through this orifice focuses the gas into a thin jet which subsequently breaks into equal-sized bubbles via a capillary instability. In a related technique, a similar arrangement was used to produce liquid droplets in air.

Microfluidic systems have been described in a variety of contexts, typically in the context of miniaturized laboratory (e.g., clinical) analysis. Other uses have been described. For example, International Patent Publication No. WO 01/89789, published Nov. 29, 2001 by Anderson et al., describes multi-level microfluidic systems that can be used to provide patterns of materials, such as biological materials and cells, on surfaces. Other publications describe microfluidic systems including valves, switches, and other components.

U.S. Patent Application Publication No. 2007/0092914 A1 describes methods for screening compounds using a compartmentalized microcapsule system.

An example of a microfluidic nanoreactor system is RainDance Technologies (RDT) Personal Laboratory System (PLS) instrument. The PLS is a type of lab-on-a-chip device. In this system, nanoreactors are micrometer size droplets containing particles of uniform and controllable size from 0.5 μm to 100 μm. Nanoreactors are prepared by the addition of surfactant materials in order to nano-aliquot the solution into discrete vesicle-like spheres. Nanoreactors are fused or split to perform a wide variety of processes including high-throughput screening techniques. See PCT WO 2007/081385, WO 2007/081386, WO 2007/081387, and WO 2007/089541.

All references cited herein, including patent applications and publications, are incorporated by reference in their entirety.

### BRIEF SUMMARY OF THE INVENTION

The invention provides methods and assays for using nanoreactors.

In one aspect, the invention provides methods in a microfluidic system for washing the contents of a nanoreactor. Such methods comprise the steps of a) fusing a first nanoreactor, containing a particle, with a second nanoreactor, containing a washing solution, to form a combined nanoreactor, wherein the diameter of the second nanoreactor is at least about two fold the diameter of the first nanoreactor; and wherein the contents of the first nanoreactor are diluted in the combined nanoreactor; b) splitting the combined nanoreactor into a plurality of nanoreactors; and c) separating the nanoreactor containing the particle from the plurality of nanoreactors formed in step b).

In another aspect, the invention provides methods for tracking a sample in a nanoreactor comprising the steps of a) fusing a sample nanoreactor comprising a sample and a reporter with a reagent nanoreactor comprising a particle and a reagent, wherein the reporter comprises a first reactive group, and a second reactive group and a reagent are associated with the particle; wherein the first reactive group reacts with the second reactive group so that the reporter is linked to the particle; and b) tracking the sample nanoreactor that has reacted with the reagent nanoreactor by tracking the nanoreactor containing the reporter.

In another aspect, the invention provides methods for measuring concentration of an analyte in a sample, said method comprising: a) compartmentalizing a sample into a plurality of nanoreactors, wherein at least about 80% of the nanoreactors contain no more than a single analyte molecule; and b) detecting the nanoreactor containing at least an analyte molecule; wherein the number of nanoreactors containing analyte molecules indicates the concentration of the analyte in the sample.

### BRIEF DESCRIPTION OF THE DRAWINGS

FIG. 1 shows the droplet (nanoreactor) reagent and sample preparations for a sandwich assay.

FIG. 2 shows the preparation of droplets (nanoreactors) containing multiple sandwich immunoassays.

FIG. 3 shows one sample assayed with multiple fluorescence depolarization immunoassays.

FIG. 4 shows coded droplet aliquots (nanoaliqouts) of clinical samples for testing with sandwich immunoassay reagents in droplets.

FIG. 5 shows combined sample droplets (nanoreactors) aliquoted clinical samples and assays. Combined sample droplets can be mixed with combined assay droplets and assayed on one droplet chip. One chip may be used for the entire study.

FIG. 6 shows a single-chip study using a single-tube of combined clinical samples and a single-tube library of assays.

FIG. 7 shows a droplet washing protocol for a sandwich immunoassay.

FIG. 8 shows a detection and collection protocol.

FIG. 9 shows detection of a desired droplet (nanoreactor) as part of a detection and collection protocol.

FIG. 10 shows collection of a desired droplet (nanoreactor) as part of a detection and collection protocol.

FIG. 11 shows collection of a second desired droplet (nanoreactor) as part of a detection and collection protocol.

FIG. 12 shows detection of an undesired droplet (nanoreactor) as part of a detection and collection protocol. The undesired droplet (nanoreactor) is returned to the starting pool.

FIG. 13 shows a sample processing, target capture and wash steps of a bDNA signal amplification assay for mRNA quantification.

FIG. 14 shows a bDNA amplifier hybridization and wash steps of a signal amplification assay for mRNA quantification.

FIG. 15 shows labeled probe hybridization, wash and detection steps of a signal amplification assay for mRNA quantification.

FIG. 16 is a schematic drawing of a process for tracking a sample in a droplet (nanoreactor) after the droplet has been combined with another droplet containing assay reagent(s).

FIG. 17 is a schematic drawing for sample-assay combination coding.

### DETAILED DESCRIPTION OF THE INVENTION

### A. General Techniques

The practice of the present invention will employ, unless otherwise indicated, conventional techniques of molecular biology (including recombinant techniques), microbiology, cell biology, biochemistry, chemistry and immunology, which are within the skill of the art.

### B. Definitions

Unless defined otherwise, all technical and scientific terms used herein have the same meaning as commonly understood by one of ordinary skill in the art (e.g., in cell culture, molecular genetics, nucleic acid chemistry, hybridization techniques and biochemistry).

As used herein, the singular form “a”, “an”, and “the” includes plural references unless indicated otherwise.

Reference to “about” a value or parameter herein includes (and describes) embodiments that are directed to that value or parameter per se. For example, description referring to “about X” includes description of “X”.

As used herein, an “analyte” is a compound, including biological molecules, that can be detected using any techniques. Examples of analytes are proteins, nucleic acids, carbohydrates, and lipids.

As used herein, an “average diameter” of a population of nanoreactors is the arithmetic average of the diameters of the nanoreactors.

As used herein, “DNA” (deoxyribonucleic acid) refers to any chain of sequence of the chemical building blocks adenine (A), guanine (G), cytosine (C) and Thymine (T), called nucleotide bases, that are linked together on a deoxyribose sugar backbone. DNA can have one strand of nucleotide bases, or two complimentary strands which may form a a double helix structure. RNA (ribonucleic acid) means any chain of chemical building blocks adenine (A), cytosine (C), guanine (G and Uracil (U), called nucleotide bases, that are linked together on a ribose sugar backbone. RNA typically has one strand of nucleotide bases.

As used herein, a “fluid” is given its ordinary meaning, i.e., a liquid or a gas. Preferably, a fluid is a liquid. The fluid may have any suitable viscosity that permits flow. If two or more fluids are present, each fluid may be independently selected among essentially any fluids (liquids, gases, and the like) by those of ordinary skill in the art, by considering the relationship between the fluids. The fluids may each be miscible or immiscible. Those of ordinary skill in the art can select suitable miscible or immiscible fluids, using contact angle measurements or the like, to carry out the techniques of the invention.

As used herein, a “nanoreactor” (used interchangeably with “droplet” or “microdroplet”) is an artificial compartment whose delimiting borders restrict the exchange of the components in a sample into the medium surrounding the nanoreactor. The delimiting borders preferably completely enclose the contents of the nanoreactor

As used herein, a “particle” means any substance that can be encapsulated within a droplet for analysis, reaction, sorting or any operation according to the invention. Particles includes, but are not limited to, microscopic beads (e.g. fluorescently labeled beads), latex, glass, silica or paramagnetic beads, dendrimers and other polymers, other porous or non-porous materials (such as quantum dots or nanobarcodes, and biomaterials such as liposomes, vesicles and other emulsions). Beads ranging in size from 0.1 micron to 1 mm can be used in the devices and methods of the invention and are therefore encompasses with the term “particle” as used herein. The devices and methods of the invention are also directed at sorting and/or analyzing molecules of any kind, including polynucleotides, polypeptides and proteins (including enzymes) and their substrates and products and small molecules (organic and inorganic). The particles are sorted and/or analyzed by encapsulating the particle into individual droplets and these droplets are then sorted, combined and/or analyzed in a microfabricated device.

Particles can have reporters (labels) and signatures (tags) that can be used to identify one particle from another. Tags can include several formats including, but not limited to quantum dots, fluorescent dyes, ratios of fluorescent dyes and/or quantum dots, radioactivity, radio tags, materials with other optical signatures, oligonucleotides, peptides and mass labeled molecules. For example, a set of beads containing two or more quantum dots in discrete amounts with an ability for detecting and differentiating the beads containing one discrete ratio from the other beads having different discrete ratios. A signature (or a tag) is a way of coding a particle (e.g. a bead).

As used herein, a “peptide” is a chain of chemical building blocks called amino acids that are linked together by chemical bonds called peptide (amide) bonds. A “protein” is a polypeptide (one or more peptides) produced by a living organism, by in vitro translation or by chemical synthesis. An “enzyme” is a polypeptide molecule, usually a protein, that catalyzes chemical reactions of other substances. The enzyme itself is not altered or destroyed upon completion of the reaction and can therefore be used repeatedly to catalyze reactions. A substrate refers to any substance upon which an enzyme acts.

As used herein “reagent” is any molecule or material that reacts or binds to another molecule, particularly a molecule in the sample. Reagents may be antibodies, aptamers, receptors, ligands, small molecules, peptides, oligonucleotides, protein nucleic acids (PNA) and fragments thereof. Reagents may also be particles such as nanomaterials that have certain absorptive/binding properties. Reagents may be general binders/reactors or have a high degree of specificity. Reagents could include more than one type of molecule such as two antibodies used in a sandwich immunoassay. For example, one component of a reagent is associated with the particle and thereby to the second reactant, for example, an antibody conjugated to a bead that has been modified with the second reactant. In some embodiments, reagents could be antibodies used for a sandwich immunoassay. In some embodiments, reagents could be oligonucleotide probes used in a PCR-based or ligation-based assay.

As used herein, a “reporter” (used interchangeably with the term “label”) is a molecule or a portion thereof, that is detectable or measurable. For example, a reporter may be detected by optical detection. The association of a reporter with a sample, particle, molecule, cell, or virion or with a particular marker or characteristic of the sample allows identification of the sample, particle, molecule, cell or viron, or the presence or absence of a characteristic of the sample, particle, molecule, cell or virion. For example, a reporter can be added to a particular sample to identify that sample. Multiple reporters can be used to label a plurality of samples with unique identifiers. For use with a sample, a reporter may be used to identify characteristics of what patient or population a sample represents. For use with molecules such as polynucleotides, a reporter may be used to identify characteristics including size, molecular weight, the presence or absence of particular constituents or moieties (such as particular nucleotide sequences or restriction sites). For use with cells, a reporter may be used to identify characteristics such as antibodies, proteins, sugar moieties, receptors, polynucleotides, and fragments thereof. Reporters include, but are not limited to, dyes, fluorescent, ultraviolet or chemiluminescent agents, chromophores, radio-labels, mass spectrometry tag molecules, resonance raman tag molecules (including surface enhanced raman spectroscopy (SERS)) or other spectral tag or other molecule that may be detected with or without some kind of stimulatory event. Fluorescent reporters can include, but are not limited to, rhodamine, fluorescein, Texas red, Cy 3, Cy 5, phycobiliprotein (e.g. phycoerytherin), green fluorescent protein, YOYO-1, PicoGreen and quantum dots. In one embodiment, the reporter is a protein that is optically detectable without a device, e.g. laser, to stimulate the reporter, such as horseradish peroxidase (HRP). A protein reporter can be expressed in the cell that is to be detected, and such expression may be indicative of the presence of the protein or it can indicate the presence of another protein that may or may not be coexpressed with the reporter. A reporter may also include any substance on or in a cell that causes a detectable reaction, for example by acting as a starting material, reactant or a catalyst for a reaction which produces a detectable product. Cells may be sorted, for example, based on the presence of the substance, or on the ability of the cell to produce the detectable product when the reporter substance is provided. In another embodiment, the reporter may be an oligonucleotide, peptide or other polymer comprised of building blocks where the identity and/or sequence of the building blocks is a unique marker. For example, a number of oligonucleotides with different sequences could be used as reporters, each specifically added to a different sample and then following reaction with various reactant droplets PCR or other oligonucleotide-based detection method used to identify which samples were mixed with which reactants.

As used herein, a “signature” (used interchangeably with the terms “marker” or “tag”) is a characteristic signal or other detectable characteristic of a reporter or a group of reporters. Signatures can be used generally or for specific labeling. For molecules, a signature can be particular constituents or moieties, such as restriction sites or particular nucleic acid sequences in the case of polynucleotides. For cells and virions, characteristics may include proteins (such as enzymes), receptors and ligand proteins, saccharides, polynucleotides, and combinations thereof, or any biological material associated with a cell or virion. The product of an enzymatic reaction may also be used as a signature. The signature may be directly or indirectly associated with the reporter or can itself be a reporter. The signature can also be created by combinations of reporters. Thus a signature is generally a distinguishing feature of a particular sample, particle, molecule, cell or virion and a reporter is generally an agent which directly or indirectly identifies or permits measurement of a signature.

As used herein, a “sample” encompasses a variety of sample types, including those obtained from an individual. The definition encompasses blood and other liquid samples of biological origin, solid tissue samples such as a biopsy specimen or tissue cultures or cells derived therefrom, and the progeny thereof. A sample can be from a microorganism (e.g., bacteria, yeasts, viruses, viroids, molds, fungi) plant, or animal, including mammals such as humans, rodents (such as mice and rats), and monkeys (and other primates). A sample may comprise a single cell or more than a single cell. The definition also includes samples that have been manipulated in any way after their procurement, such as by treatment with reagents, solubilization, or enrichment for certain components, such as proteins or polynucleotides. The term “sample” encompasses a clinical sample, and also includes cells in culture, cell supernatants, cell lysates, serum, plasma, biological fluid, human tissue propagated in animals, and tissue samples. Examples of a sample include blood, plasma, serum, urine, stool, cerebrospinal fluid, synovial fluid, amniotic fluid, saliva, lung lavage, semen, milk, nipple aspirate, prostatic fluid, mucous, and tears.

A “small molecule” as used herein, is meant to refer to a molecule that has a molecular weight of less than about 5 kD and most preferable less than about 1 kD. Small molecules can be, e.g., nucleic acids, peptides, peptides, peptidomimetics, carbohydrates, lipids, metabolites or other organic or inorganic molecules. Libraries of chemical and/or biological mixtures, such as clinical, fungal, bacterial or algal extracts, are known in the art.

As used herein, a “switch” is a mechanism using any physical force to divert, steer, or direct droplets as desired. A switch can be electrical, mechanical, or other.

As used herein, “tracking” can include following, tracing or monitoring the origins, pathways, identity, and associated data of a nanoreactor, or components of a nanoreactor. For example, tracking can include monitoring the combination of a particular sample droplet with a particular reagent droplet. In another example, tracking of a nanoreactor clinical sample can include tracking of the patient data associated with that clinical sample.

### C. Methods for Nano-Aliquoting and Coding of Samples

The present invention provides methods of nano-aliquoting and coding a sample, including the step of compartmentalizing a mixture of a sample and a coded molecule into a nanoreactor.

In some embodiments, the quantity of the sample in the nanoreactor is sufficient for detection of an analyte in the sample, and the quantity of the coded molecule in the nanoreactor is sufficient for identification of the nanoreactor. In some embodiments, a plurality of samples are nano-aliquoted and coded. Separate mixtures are compartmentalized into separate nanoreactors such that each mixture comprises a sample and a coded molecule. The quantity of each sample in a nanoreactor is sufficient for detection of an analyte in the sample, and the quantity of the coded molecule in the nanoreactor is sufficient for identification of the nanoreactor. Separate nanoreactors are pooled into a collection of nanoreactors.

In some embodiments, a sample is compartmentalized into a plurality of nanoreactors wherein at least about 80% of the nanoreactors contain no more than a single analyte molecule.

Samples used in the present invention can be derived from a wide variety of sources. Samples can include biological samples, chemical samples and synthetic samples. For example, the sample may be a clinical sample. Examples of clinical samples include cells, cells in culture, cell supernatants, cell lysates, serum, plasma, biological fluids, human tissue propagated in animals and tissue samples. Other samples include but are not limited to components derived from biological samples or produced synthetically such as proteins, polynucleotides, carbohydrates and lipids.

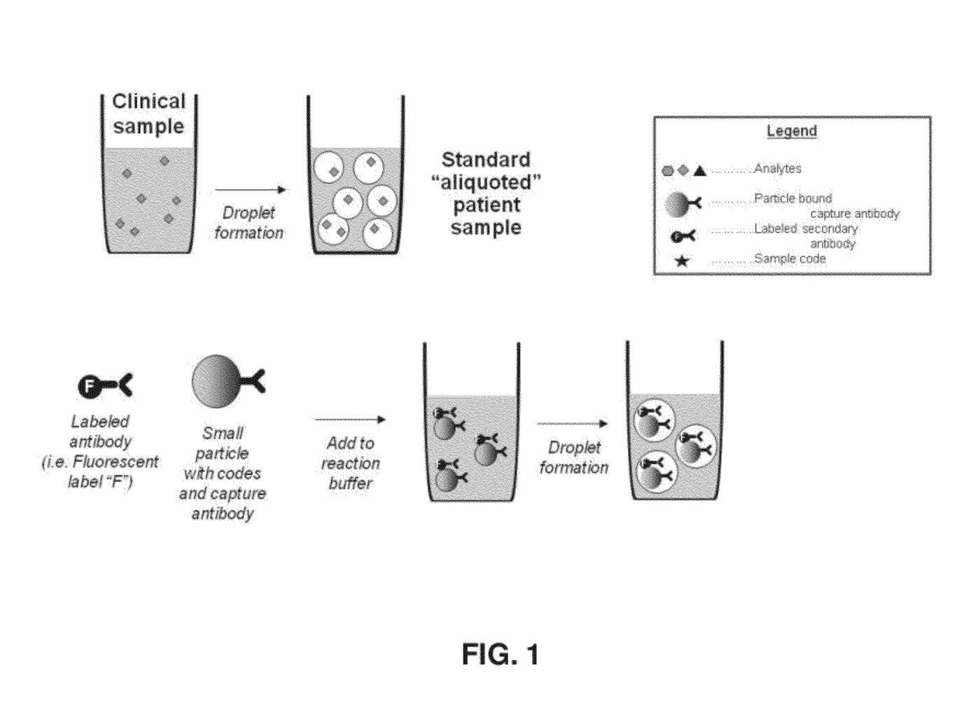

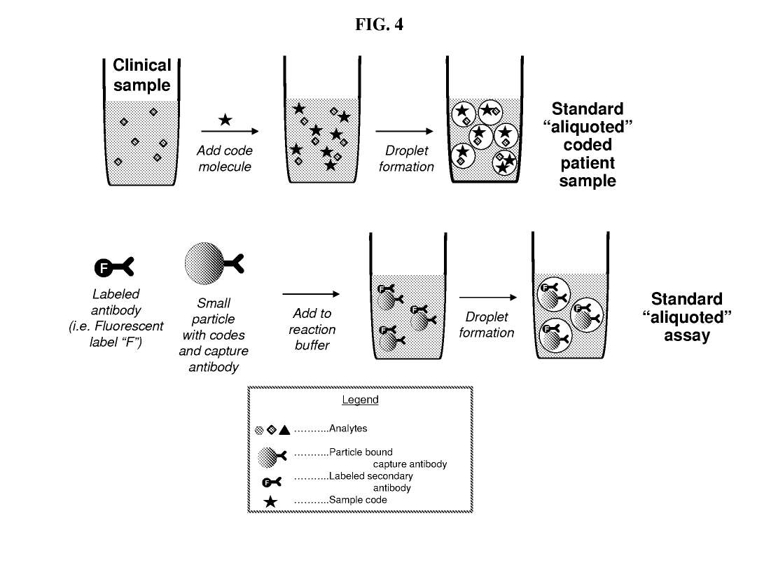

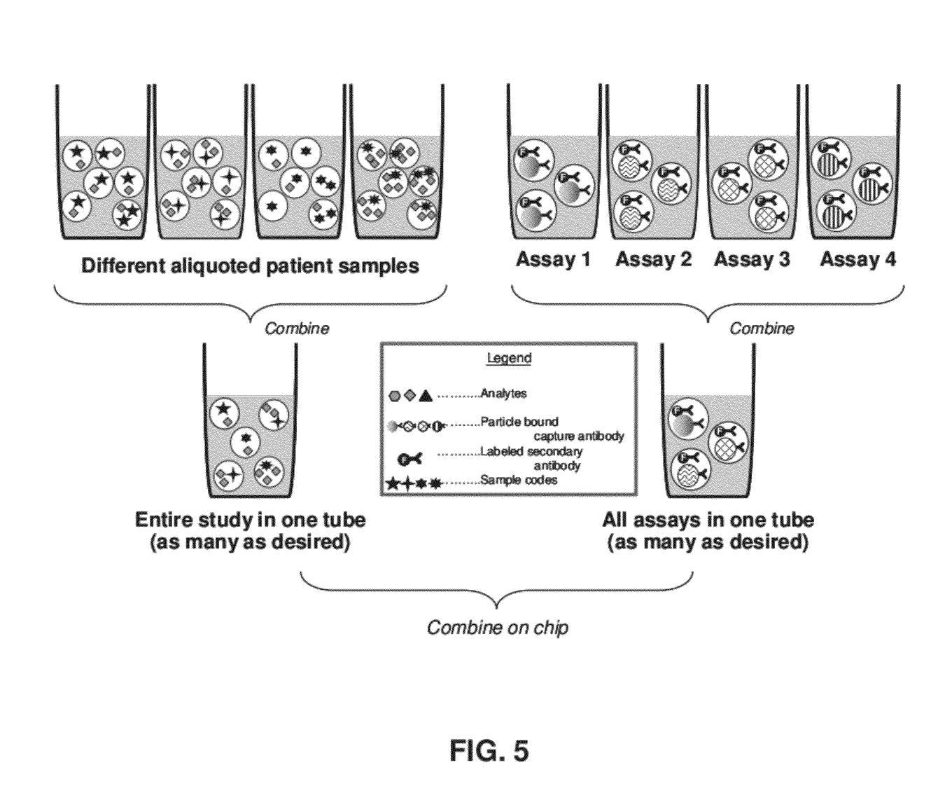

To nano-aliquot samples into sample nanoreactors, a sample of interest (such as a serum sample from a patient in a large observational study) is mixed with a specifically coded molecule. The coded material may be dye coded beads like those used for the solid phase in a sandwich assay. The beads are added in sufficient quantity to permit at least one bead per nanoreactor (FIGS. 1 and 4). The sample and the coded material are allowed to be incorporated into nanoreactors; for example, by methods described herein and by methods known in the art. Sizes and volumes may be adjusted. Likewise, the quantity of coded molecule may be adjusted to allow for identification of the nanoreactor. By using a specific code for a specific sample, multiple samples can be combined and tested in the same assay system. For example, samples from different patients are aliquoted into nanoreactors such that each patient sample has its own code. Samples are then combined to form a pool of assay samples that can be analyzed together (FIG. 5). As such, it is possible to test all samples from a study at the same time under the same assay reaction conditions.

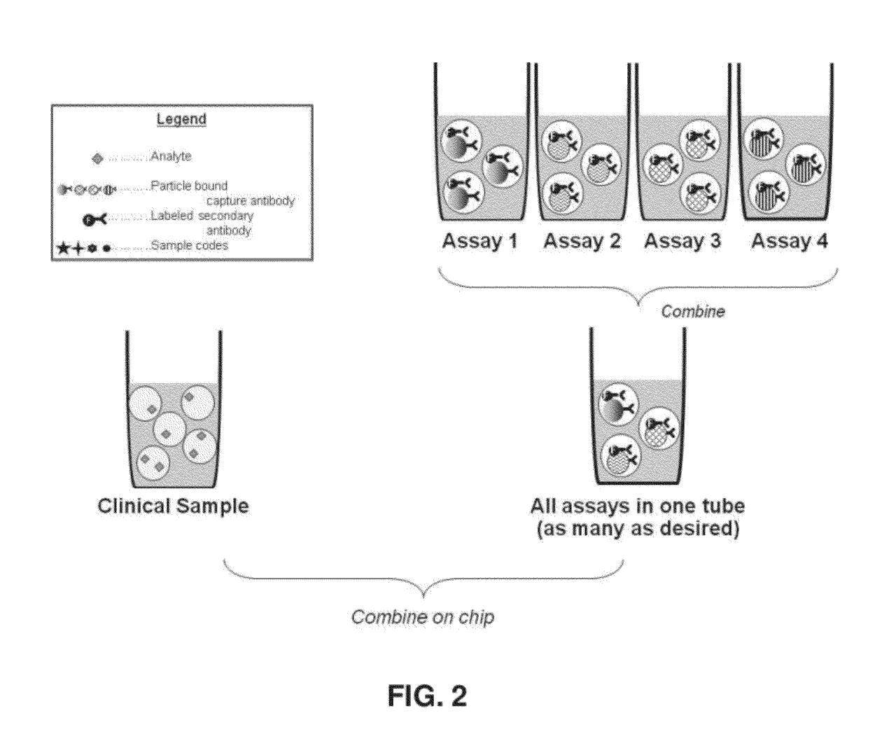

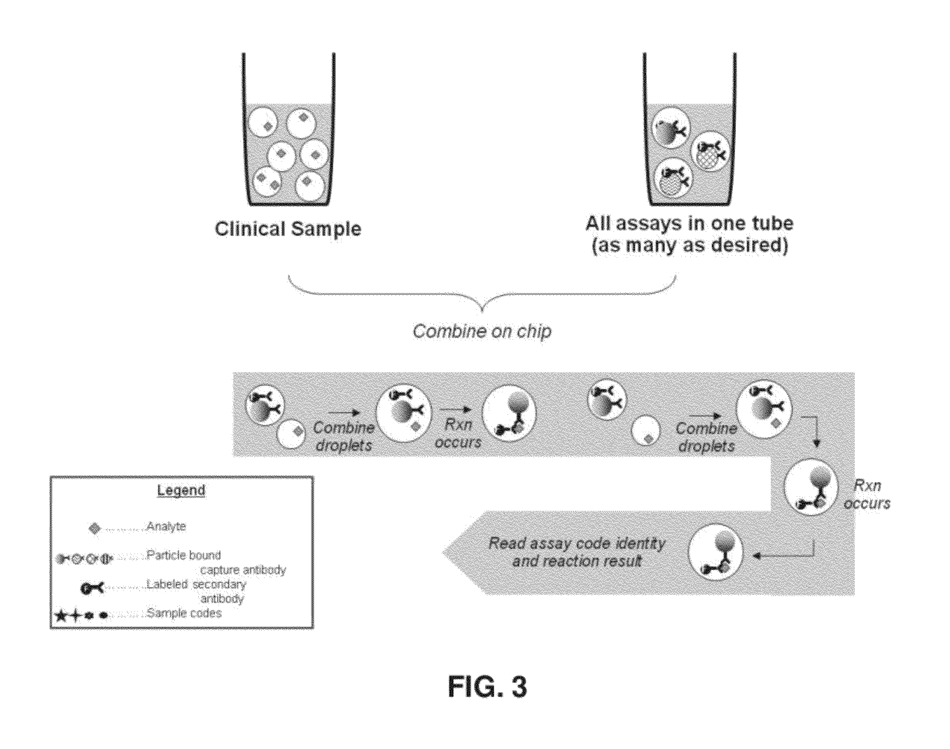

Assay or reagent nanoreactors can be formed in a manner similar to sample nanoreactors. Assay reagents are mixed with a specifically coded molecule and then incorporated into a nanoreactor; for example by any of the methods described herein. Multiple assay or reagent nanoreactors can be formed such that each assay has its own code; see FIG. 2, for example. The amount of depolarization is indicative or the quantity of substrate bound to the solid phase. Assay nanoreactors can be combined to test multiple parameters in a single sample as long as codes for different assay reactions are distinct (e.g., as shown in FIG. 3). In addition, combined assay nanoreactors may be used with combined sample nanoreactors to analyze multiple parameters from multiple samples in nanoreaction system (e.g., as shown in FIG. 5).

The nanoreactors can be coded in a variety of ways for future identification and selection. For example, a coded molecule is incorporated into the sample and/or the assay nanoreactors (e.g., as shown in FIG. 4). Any coded molecules (such as fluorescent tags, nano-bar code, dye coded beads, and quantum dots) that can be detected may be use. Czarnik, A. W. (1997) Curr Opin Chem Biol 1:60-66; Han, M., et al. (2001) Nat Biotechnol 19:631-635. The nanoreactors may then be sorted (e.g. by using a fluorescence activated cell sorter—FACS) based on the code molecules in the nanoreactors.

Nanoreactors can be optically tagged by, for example, incorporating fluorochromes. In a variation, the nanoreactors are optically tagged by incorporating quantum dots: quantum dots of 6 colors at 10 concentrations would allow the encoding of 10⁶ nanoreactors (Han, M., et al. (2001) Nat Biotechnol 19:631-635).

A fluorescent dye can be used for labeling the detection component of assays in various configurations. For example, sandwich style immunoassays or nucleic acid assays can be constructed using energy transfer, fluorescence depolarization and other methods. The present invention provides for a means to conduct heterogeneous assays such as direct fluorescent label detection in a standard fluorescent immunoassay through a nanoreactor form of washing. The “coding space”, wavelengths for fluorescent coding of nanoreactors, and the “labeling space”, wavelengths for fluorescent detection, should be well separated in the useful light spectrum.

Fluorescence may be enhanced by the use of Tyramide Signal Amplification (TSA™) amplification to make the microbeads fluorescent (Sepp, A., et al. (2002) FEBS Letters 532:455-458). In this system, peroxidase (linked to another compound) binds to the microbeads and catalyzes the conversion of fluorescein-tyramine in to a free radical form which then reacts locally with the microbeads. Methods for performing TSA are known in the art, and kits are available commercially (NEN). TSA may be configured such that it results in a direct increase in the fluorescence of the microbeads, or such that a ligand is attached to the microbeads which are bound by a second fluorescent molecule, or a sequence of molecules, one or more of which is fluorescent.

Nanoreactors or beads can also be identified by Nanobarcodes™ (Oxonica). Nanobarcodes have been previously described, (for example U.S. Pat. No. 7,225,082, reference incorporated herein). Nanobarcodes are particles comprising a plurality of segments which result in their diversity. For example, a bar code with nine segments comprised of four materials will have a complexity of 4⁹, and therefore can provide >260,000 unique barcodes. A variety of different methods may be used to detect nanobarcodes including but not limited to optical detection systems, scanning probe techniques, electron beam techniques, electrical detection mechanisms, mechanical detection mechanisms and magnetic detection mechanisms.

In some embodiments, the nanoreactors used in the method of the present invention are capable of being produced in very large numbers, and thereby to compartmentalize a library of samples or compounds. Optionally, each nanoreactor may contain a different coded molecule for identification of each nanoreactor. The nanoreactors used herein allow mixing, splitting, and sorting to be performed thereon, in order to facilitate the high throughput potential. In some embodiments, nanoreactors can be a droplet of one fluid in a different carrier fluid, where the confined components are soluble in the droplet but not in the carrier fluid. In some embodiments, there is another material defining a wall, such as a membrane (e.g., in the context of lipid vesicles; liposomes) or non-ionic surfactant vesicles, or those with rigid, nonpermeable membranes, or semipermeable membranes.

In some embodiments, the diameters of the nanoreactors are ranging from about 5 to about 100 micrometers. Those of ordinary skill in the art will be able to determine the average diameter of a population of nanoreactors, for example, using laser light scattering or other known techniques.

Methods of forming and handling nanoreactors are known and are further described under Section H herein.

### D. Methods for Washing Nanoreactors

In some embodiments, the present invention provides methods of washing nanoreactors containing a particle in a microfluidic system. In one variation a nanoreactor containing a particle is fused with a second nanoreactor containing a washing solution to form a combined nanoreactor. In this example, the diameter of the second nanoreactor is at least about two fold the diameter of the first nanoreactor. In some embodiments the diameter of the second nanoreactor is about five fold the diameter of the first nanoreactor. In some embodiments the diameter of the second nanoreactor is about ten fold the diameter of the first nanoreactor. The combined nanoreactor is split into a plurality of nanoreactors and the nanoreactors containing the particle may be separated from the plurality of nanoreactors.

In some embodiments a nanoreactor containing a wash solution is fused with a fused sample/assay nanoreactor (the reaction nanoreactor) to remove unwanted components; for example, excess labeled substrate. Typically, a wash nanoreactor is larger than a reaction nanoreactor in order to dilute the components of the reaction nanoreactor. In some cases, the wash nanoreactor contains components to dissociate components in a nanoreactor. A wash nanoreactor is fused with a reaction nanoreactor, the newly combined nanoreactor is split such that the amount of unwanted components in the reaction nanoreactor is reduced. The resulting washed nanoreactor may be further processed. For example, it may be collected and analyzed, fused with a different assay nanoreactor for subsequent analysis or being further washed.

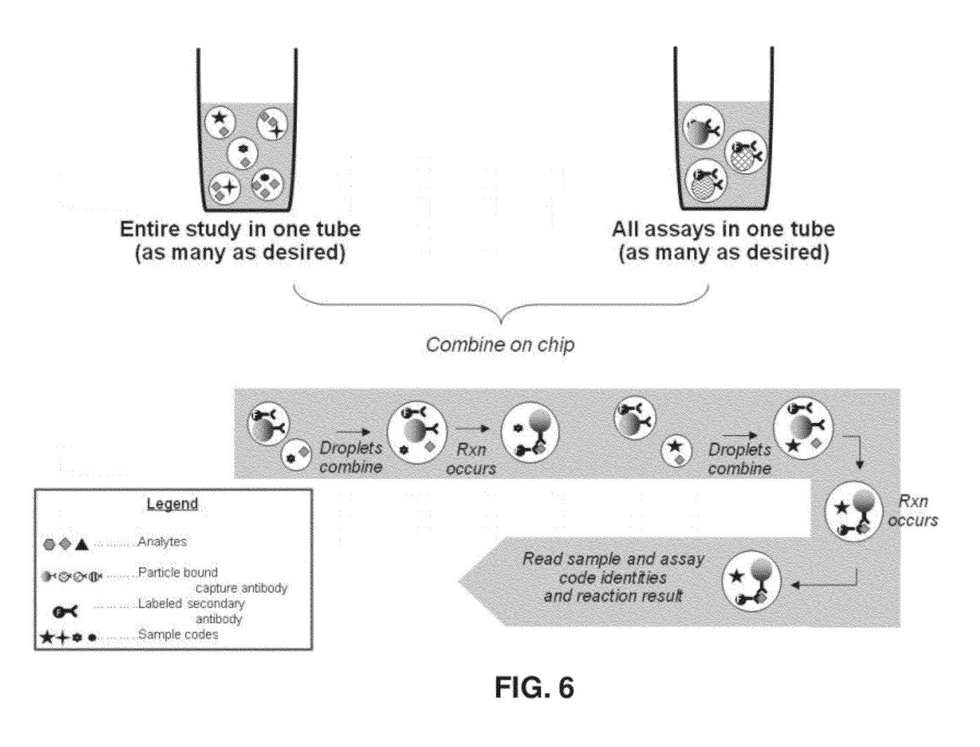

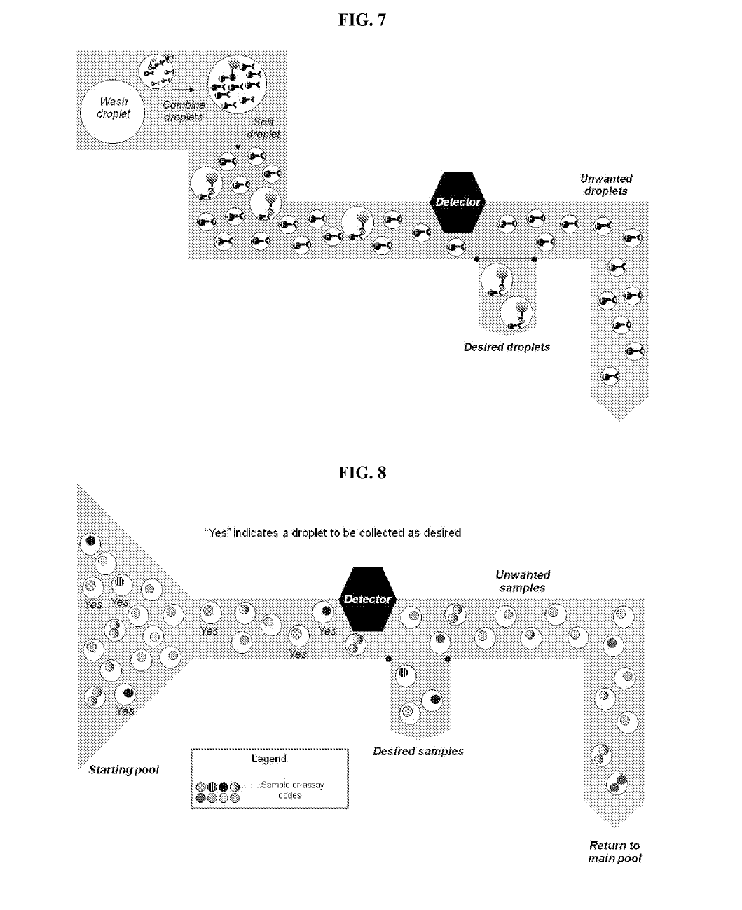

A wash step can be used in a variety of processes using nanoreactors. For example, a wash step may be included in a simple sandwich immunoassay conducted in nanoreactors as shown in FIGS. 6 and 7. In this particular example, sandwich assay is conducted in nanoreactors as shown in FIG. 6. Sample nanoreactors containing beads with specific sample codes are fused with assay nanoreactors containing a coded bead or small particle joined to a capture antibody and an excess of fluorescent labeled antibodies. Nanoreactors can then be fused as described under Section H with a larger nanoreactor containing a wash reagent to dilute but not destabilize the sandwich complex. In some cases, components of the nanoreactor are crosslinked prior to wash steps if the off-rate of the sandwich assay component is problematic. The new and larger nanoreactor contains the solid phase and the unused labeled antibody in diluted form. Nanoreactors can then be split into a series of smaller nanoreactors as described herein. Washed nanoreactors can then be passed by a detector for selection and collection. Only reactors containing the coded bead are collected while other nanoreactors may be discarded; for example; nanoreactors containing unreacted fluorescently labeled antibodies. Additional wash steps may be carried out with collected nanoreactors such as rewashing if the dilution of assay components prior to detection is insufficient. A ten-fold increased wash nanoreactor diameter relative to assay nanoreactors results in a 1000-fold dilution in reactants. If one particle is insufficient for the final detection step, nanoreactors containing the same coded particle may be combined.

### E. Methods for Collecting of Desired Nanoreactors

In some embodiments, the present invention also provides methods of selecting desired nanoreactors from a collection of nanoreactors in a microfluidic system, comprising the steps of: a) detecting the desired nanoreactors from a collection of nanoreactors flowing in a microfluidic system based on the coded molecules in the nanoreactors; b) separating the desired nanoreactors from the undesired nanoreactors; and c) returning the undesired nanoreactors to a starting pool through a microfluidic system.

The present invention also provides methods of selecting desired nanoreactors from a collection of nanoreactors in a microfluidic system, comprising the steps of: a) separating the desired nanoreactors from the undesired nanoreactors, wherein the desired nanoreactors are detected from a collection of nanoreactors flowing in a microfluidic system based on the coded molecules in the nanoreactors; and b) returning the undesired nanoreactors to a starting pool through a microfluidic system.

The process achieves the selective use of nanoreactors from complex mixtures including highly complex mixtures without significant loss (without unacceptable loss) of the unwanted nanoreactors in the mixture. This is useful for any complex mixture (e.g., assay mixtures as in FIG. 1), including specific reagents such as particular pH buffers, salts, detergents, antibodies, standard proteins, probes, dyes, etc. Only the nanoreactors containing desired components may be used. Using this approach, the number and type experiments that could be conducted are limited only by the imagination of the experimenter.

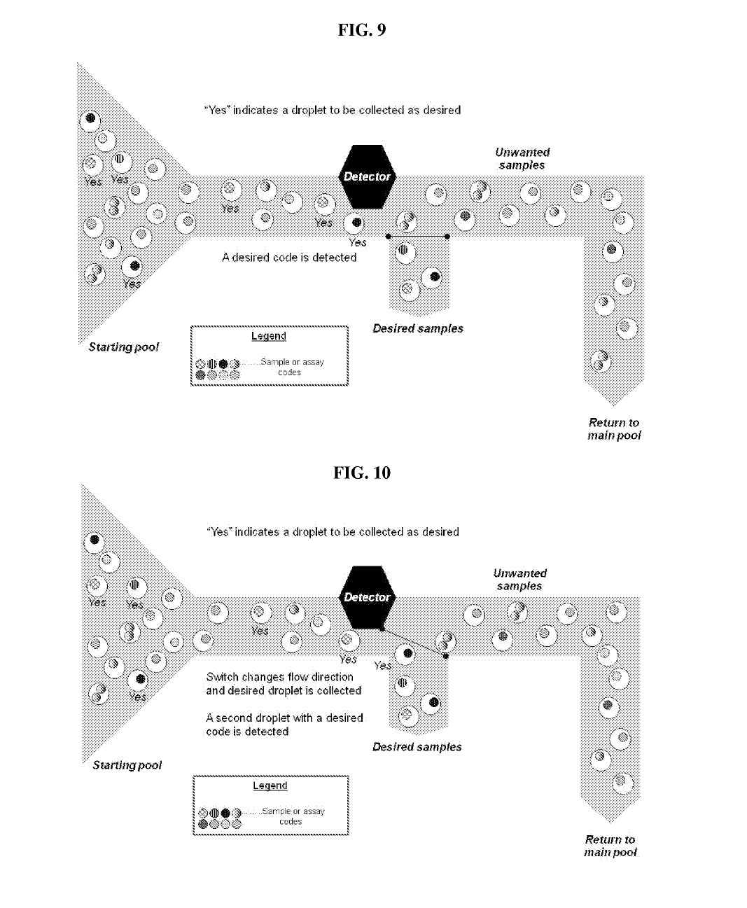

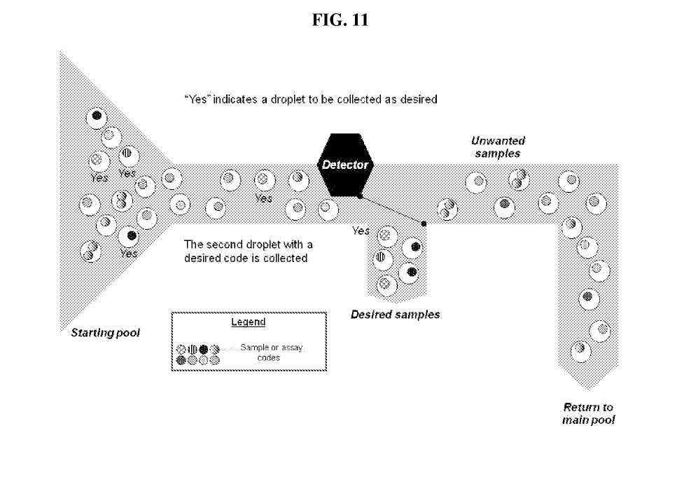

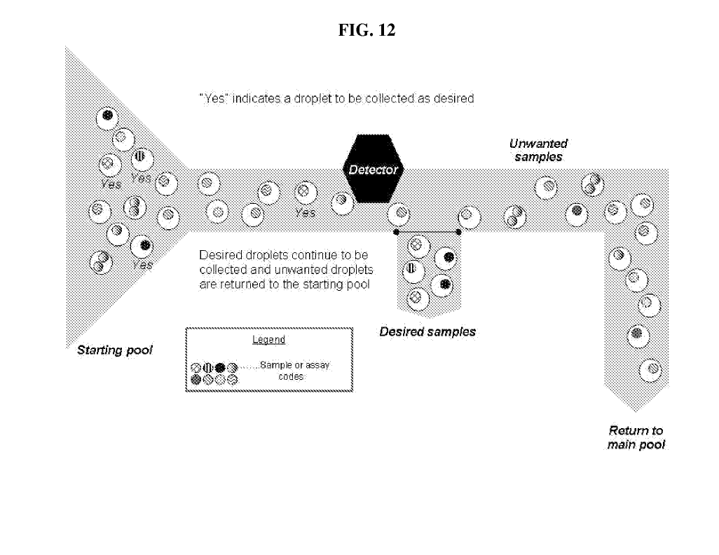

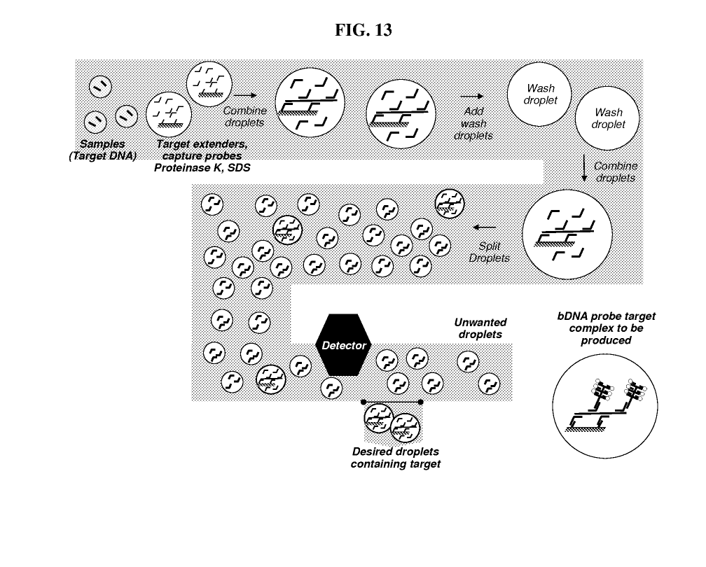

The nanoreactors of the present invention can be coded such that sample nanoreactors from individual samples have a unique identifier. Likewise, assay nanoreactors can be coded such that each assay nanoreactor has a unique identifier. Unique identifiers can be programmed into microfluidic systems such that only specific nanoreactors are recorded for a particular set of operations. In addition, microfluidic systems can be programmed such that specific codes can be used to trigger events such as collection. For example, a microfluidic system can be programmed in a manner similar to the way that stained cells are collected on a flow cytometer. An example of a method of collecting desired nanoreactors without the loss of unwanted nanoreactors is shown in FIGS. 8-12. Any screening and collection methods known may be used to segregate desired nanoreactors from unwanted nanoreactors. In this particular example, a detector interacts with a switch to segregate desired nanoreactors from undesired nanoreactors (FIG. 8). When a desired nanoreactor is detected (FIG. 9), a switch is activated and the desired nanoreactor is collected; for example, in a collection channel (FIG. 10). When an undesired nanoreactor is detected, the switch is activated to divert the undesired nanoreactor away from the desired nanoreactors; for example, in a second channel (FIG. 12). The undesired nanoreactors do not have to be discarded but can be returned to the starting pool or collected for future analysis.

An important consideration is that the coded nanoreactors that are returned to the original pool can be used again, perhaps a very large number of times. This necessitates that the codes be sufficiently stable for detection. This can be problematic for fluorescent dyes since many may bleach during storage or detection with strong light. Stable dyes, such as quantum dots that do not bleach significantly, may be used. Alternatively, true bar coded nano-materials can be used with optical detector selection.

With many uses of the complex mixtures some of the components could become limiting in concentration. It could be important or desired to monitor the composition of the mixtures and replenish missing components or discard the mixture.

The methods of the present invention may be used to conduct heterogeneous assays such as direct fluorescent label detection in a standard fluorescent immunoassay through a nanoreactor form of washing.

### F. Methods for Tracking Sample Droplets

The invention also provides methods for tracking a sample in a nanoreactor comprising the steps of a) fusing a sample nanoreactor comprising a sample and a reporter with a reagent nanoreactor comprising a particle and a reagent, wherein the reporter comprises a first reactive group, and a second reactive group and a reagent are associated with the particle; wherein the first reactive group reacts with the second reactive group so that the reporter is linked to the particle; and b) tracking the sample nanoreactor that has reacted with the reagent nanoreactor by tracking the nanoreactor containing the reporter.

In some embodiments, the present invention involves the reaction between a first and second reactive group such that the reporter from the sample is linked (covalently or non-covalently) to the particle in the reagent nanoreactors. Any chemical reaction using two or more reactants compatible with the medium, other molecules present and other conditions present could be used and should be apparent to those skilled in the art of organic, chemistry, conjugation chemistry and biochemistry. For example, acylation chemistries such as reactions between nucleophiles (such as amines, hydroxyls, thiols and hydrazines) with acyl donors (such as activated esters, including are but not limited to hemi-succinate esters of N-hydroxysuccinimide, sulfo-N-hydroxysuccinimide, hydroxybezotriazoles and p-nitrophenol, esters, anhydrides, acid halides and thiol esters) to form amides, esters, thioesters and hydrazides may be used. Other examples are: 1) condensation reactions between nucleophiles (such as amine, hydrazines and alkoxyamines) with carbonyl compounds (such as ketones and aldehydes) to form immines, hydrazones and oximes, nucleoplhiles such as thiols with electrophillic acceptors such as maleimides and alpha-halo carbonyls to product sulfo-ethers; diene and dienophiles to product cyclohexadiene products via Diels-Alder cycloadditions (including heterop version thereof), alkynes (particularly terminal alkynes) and azides to produce triazoles via [3+2] dipolar cycloadditions; boronic acids or esters with aryl or vinyl halides, particularly iodides to form biaryl products for example with two aryl reactants via transition metal catalyzed cross-coupling reactions.

Non-covalent product could be formed if the first and second reactants were ligand-receptor pairs such as streptavidin or biotin. Particularly useful are those reactions, such as the Diels-Alder cycloaddition and dipolar cycloaddition, that have very limited or no cross reactivity with the other components of the droplets. In another embodiment, the first and second reactants could be substrates for an enzyme whereby the reaction catalyzed by the enzyme links the reporter and the particle. In another embodiment, the first and second reactants may be linked via a hetero- or a homo-bifunctional crosslinker such as those commercially available. For example, the first and second reactants may be chosen from a list of complimentary functional groups or molecules known to participate in a particular chemical reaction or binding event. Complimentary functional groups as used herein means chemically reactive groups that react with one another with high specificity (i.e. the groups are selective for one another and their reaction provides well-defined products in a predictable fashion) to form new covalent or non-covalent bonds.

The first and second reactants can be attached to the reporter and particle, respectively, using any chemistry apparent to those skilled in the art.

A linker may be used between the first reactant and the reporter molecule and between the particle and the second reactant. A “linker” as used herein refers to a chain comprising 1 to 100 atoms but typically less than 20 and may be comprised of the atoms or groups such as C, —NR—, O, S, —S(O)—, S(O)2—, CO, —C(NR)—, and the like, and where in R is H or is selected from the group consisting of alkyl, cycloakyl, aryl, heteroaryl, amino, hydroxyl, alkoxy, aryloxy, heteroaryloxy, each substituted or unsubstituted. The linker chain may also comprise part of a saturated, unsaturated or aromatic ring, including polycyclic and heteroaromatic rings.

“Alkyl” refers to a hydrocarbon chain typically ranging from about 1 to 20 atoms in length. Such hydrocarbon chains may be branched or straight chain, although typically straight chain is preferred. Exemplary alykyl groups include ethyl, propyl, butyl, pentyl, 1-methylbutyl, 1-ethylpropyl, 3-methylpentyl and the like. As used herein, alkyl includes cycloakyl when three of more carbon atoms are referenced.

“Aryl” refers to one or more aromatic rings, each of 5 or 6 core carbon atoms. Aryl includes multiple aryl rings that may be fused as in naphyl or unfused asin biphenyl. Aryl rings may also be fused or unfused with one or more cyclic hydrocarbons, heteroaryl or hetercyclic rings. As used herein “aryl” include heteroaryl.

Other reagents that facilitate the coupling of organic drugs and peptides to various ligands may be used. See Haitao, et al., Organ Lett. 1:91-94, 1999; Albericio, et al., J. Organic Chemistry 63:9678-9783, 1998; Arpicco, et al., Bioconjugate Chem. 8:327-337, 1997; Frisch et al., Bioconjugate Chem. 7:180-186, 1996; Deguchi, et al., Bioconjugate Chem. 10:32-37, 1998; Beyer et al., J. Med. Chem. 41:2701-2708;, 1998; Driven, et al., Chem. Res. Toxicol. 9:351-360, 1996; Drouillat, et al., J. Pharm Sci. 87:25-30, 1998; Trimble, et al., Bioconjugate Chem. 8:416-423, 1997. Chemicals, reagents and techniques useful in drug cross-linking and peptide conjugation may be used. These techniques are disclosed in general texts well known to those skilled in the art. See Dawson, et al. (Eds.), Data for Biochemical Research, 3^{rd} Ed., Oxford University Press, Oxford, UK, 1986, pp. 580; King, (Ed.), Medicinal Chemistry: Principles and Practice, Royal Society of Chemistry, Cambridge, UK, 1994, pp. 313; Shan and Wong, (Eds.), Chemistry of Protein Conjugation and Cross-Linking, CRC Press, Boca Raton, 1991, pp. 328. Additional chemical coupling agents are described in U.S. Pat. No. 5,747,641.

### G. Methods of Measuring Analyte Concentration in Samples

The invention also provides methods of measuring concentration of an analyte in a sample, said method comprising: a) compartmentalizing a sample into a plurality of nanoreactors, wherein at least about 80% of the nanoreactors contain no more than a single analyte molecule; and b) detecting the nanoreactor containing at least an analyte molecule; wherein the number of nanoreactors containing analyte molecules indicates the concentration of the analyte in the sample.

The methods may be used to measuring low concentration analyte a sample (for example, from 5×10⁻⁸ ng/ml to 5×10⁻³ ng/ml). Samples (e.g., clinical samples) are aliquoted into nanoreactors or droplets described herein. Abundant analytes are found in multiple nanoreactors bur low concentration analytes are only in some of the nanoreactors. Samples may be aliquoted into a plurality of nanoreactors wherein at least about 80% of the nanoreactors contain no more than a single analyte molecule. Concentration of the analyte in the sample may be measured by counting the number of nanoreactors containing one or more analyte molecules. In some embodiments, at least about 85%, at least about 90%, at least about 95%, or greater than 95% of the nanoreactors contain no more than a single analyte molecule. Technology for single molecule measurements is known in the art. See, e.g., WO 2006/036182.

In some embodiments, the analyte containing nanoreactors are labeled with reporters before detection. For example, samples in nanoreactors are allowed to react with an antibody comprising a reporter. Then the nanoreactors containing the sample are fused with nanoreactors containing a washing solution. The fused nanoreactors are slit into a plurality of nanoreactor, wherein at least about 80% of the nanoreactor contain no more than a single analyte molecule bound to the antibody with a label. The number of nanoreactors containing labeled analyte molecules is used to determine the concentration of the analyte.

### H. Methods for Forming and Handling Nanoreactors

Formation of Nanoreactors

Any technology that may be used to form nanoreactors may be used to compartmentalize a sample and a coded molecule. Formation of nanoreactors has been described previously, see for example U.S. Pat. Nos. 7,329,545 and 6,911,132; U.S. Patent Application Publication Nos. US 2007/0092914 A1, US 2007/0003442 A1, US 2006/0078893, US 2006/0078888 A1, and WO 2007/030501 A2, which are incorporated herein by reference.

The nanoreactors of the present invention require appropriate physical properties to allow the working of the invention. The contents of each nanoreactor may be isolated from the contents of the surrounding nanoreactors, so that there is no or little exchange of compounds. The permeability of the nanoreactors may be adjusted such that reagents may be allowed to diffuse into and/or out of the nanoreactors if desired. The formation and the composition of the nanoreactors advantageously do not abolish the activity of the target.

A wide variety of microencapsulation procedures are available and may be used to create the nanoreactors used in accordance with the present invention. See Benita, S. (ed.). (1996) Microencapsulation: methods and industrial applications. Marcel Dekker, New York.; Finch, C. A. (1993) Spec. Publ.-R. Soc. Chem., 138:35.

Nanoreactors can be generated by interfacial polymerization and interfacial complexation (Whateley, T. L. (1996) In Benita, S. (ed.), Microencapsulation: methods and industrial applications. Marcel Dekker, New York, pp. 349-375). Nanoreactors of this sort can have rigid, nonpermeable membranes, or semipermeable membranes.

Non-membranous microencapsulation systems based on phase partitioning of an aqueous environment in a colloidal system may also be used. For example nanoreactors may be formed from emulsions; heterogeneous systems of two immiscible liquid phases with one of the phases dispersed in the other as droplets of microscopic or colloidal size (Becher, P. (1957) Emulsions: theory and practice. Reinhold, N.Y.; Sherman, P. (1968) Emulsion science. Academic Press, London; Lissant, K. J. (ed.) (1974) Emulsions and emulsion technology. Marcel Dekker, New York; Lissant, K. J. (ed.). (1984) Emulsions and emulsion technology. Marcel Dekker, New York; Griffiths et al., Trends in Biotech. 24:395-402, 2006; Kelly et al., Chem. Commun. 14:1773-1788, 2007).

Emulsions may be produced from any suitable combination of immiscible liquids. For example, an emulsion may comprise water, containing the biochemical components, in the form of finely divided droplets (the disperse, internal or discontinuous phase) and a hydrophobic, immiscible liquid, such as an oil, as the matrix in which these droplets are suspended (the nondisperse, continuous or external phase). Such emulsions are termed “water-in-oil”.

The emulsion may be stabilized by addition of one or more surface-active agents (surfactants). These surfactants are termed emulsifying agents and act at the water/oil interface to prevent, or at least delay, separation of the phases. Many oils and many emulsifiers can be used for the generation of water-in-oil emulsions; a recent compilation listed over 16,000 surfactants, many of which are used as emulsifying agents (Handbook of Industrial Surfactants: An International Guide to more than 21,000 Products by Trade Name, Composition, Application, and Manufacturer, Ash, M and Ash, I. (eds) (1993) Aldershot, Hampshire, England). Suitable oils include light white mineral oil and decane. Suitable surfactants include: non-ionic surfactants (Schick, M. J. (1966) Nonionic surfactants, Marcel Dekker, New York) such as sorbitan monooleate (Span™ 80; ICI), sorbitan monostearate (Span™ 60; ICI), polyoxyethylenesorbitan monooleate (Tween™ 80; ICI), and octylphenoxyethoxyethanol (Triton X-100); ionic surfactants such as sodium cholate and sodium taurocholate and sodium deoxycholate; chemically inert silicone-based surfactants such as polysiloxane-polycetyl-polyethylene glycol copolymer (Cetyl Dimethicone Copolyol) (e.g. Abil™ 90; Goldschmidt); and cholesterol.

In some embodiments, emulsions with a fluorocarbon (or perfluorocarbon) continuous phase (Krafft, M. P., et al. (2003) Curr. Op. Colloid Interface Sci., 8:251-258; Riess, J. G. (2002) Tetrahedron, 58:4113-4131) may be utilized. For example, stable water-in-perfluorooctyl bromide and water-in-perfluorooctylethane emulsions can be formed using F-alkyl dimorpholinophosphates as surfactants (Sadtler, V. M., et al. (1996) Angew. Chem. Int. Ed. Engl., 35:1976-1978). Non-fluorinated compounds are essentially insoluble in fluorocarbons and perfluorocarbons (Curran, D. P. (1998) Angew Chem Int Ed, 37:1174-1196; Hildebrand, J. H. and Cochran, D. F. R. (1949) J. Am. Chem. Soc., 71:22; Hudlicky, M. (1992) Chemistry of Organic Fluorine Compounds, Ellis Horwood, N.Y.; Scott, R. L. (1948) J. Am. Chem. Soc., 70:4090; Studer, A., et al. (1997) Science, 275:823-826) and small drug-like molecules (typically <500 Da and Log P<5) (Lipinski, C. A., et al. (2001) Adv Drug Deliv Rev, 46:3-26) are compartmentalized very effectively in the aqueous nanoreactors of water-in-fluorocarbon and water-in-perfluorocarbon emulsions--with little or no exchange between nanoreactors.

Creation of an emulsion generally requires the application of mechanical energy to force the phases together. There are a variety of ways of doing this which utilize a variety of mechanical devices, including stirrers (such as magnetic stir-bars, propeller and turbine stirrers, paddle devices and whisks), homogenizers (including rotor-stator homogenizers, high-pressure valve homogenizers and jet homogenizers), colloid mills, ultrasound and ‘membrane emulsification’ devices (Becher, P. (1957) Emulsions: theory and practice. Reinhold, N.Y.; Dickinson, E. (1994) Emulsions and droplet size control, In Wedlock, D. J. (ed.), Controlled particle, droplet and bubble formation. Butterworth-Heinemann, Oxford, pp. 191-257), and microfluidic devices (Umbanhowar, P. B., et al. (2000) Langmuir, 16:347-351).

Nanoreactor size will vary depending upon the precise requirements of any individual screening process that is to be performed according to the present invention. In all cases, there may be an optimal balance between the size of the compound library and the sensitivities of the assays to determine the identity of the compound and target activity. In some embodiments, the average cross-sectional dimension of the nanoreactors are from about 1 microns to about 100 microns. In some embodiments, the nanoreactors have a cross-sectional dimension of less than about 100 microns, less than about 50 microns, less than about 30 microns, less than about 10 microns, less than about 5 microns, and less than about 3 microns.

The size of emulsion nanoreactors may be varied simply by tailoring the emulsion conditions used to form the emulsion according to requirements of the screening system.

Water-in-oil emulsions can be re-emulsified to create water-in-oil-in water double emulsions with an external (continuous) aqueous phase. These double emulsions can be analyzed and, optionally, sorted using a flow cytometer (Bernath, K., et al. (2004) Anal Biochem, 325:151-157).

An electric field may be applied to fluidic droplets to cause the droplets to experience an electric force. In some cases, electric charge may be created on a fluid surrounded by a liquid, which may cause the fluid to separate into individual droplets within the liquid. The fluid and the liquid may be present in a channel, e.g., a microfluidic channel, or other constricted space that facilitates application of an electric field to the fluid (which may be “AC” or alternating current, “DC” or direct current etc.), for example, by limiting movement of the fluid with respect to the liquid. Thus, the fluid can be present as a series of individual charged and/or electrically inducible droplets within the liquid. In some cases, the electric force exerted on the fluidic droplet may be large enough to cause the droplet to move within the liquid. In some cases, the electric force exerted on the fluidic droplet may be used to direct a desired motion of the droplet within the liquid, for example, to or within a channel or a microfluidic channel.

Electric charge may be created in the fluid within the liquid using any suitable technique, for example, by placing the fluid within an electric field (which may be AC, DC, etc.), and/or causing a reaction to occur that causes the fluid to have an electric charge, for example, a chemical reaction, an ionic reaction, a photocatalyzed reaction, etc. Techniques for producing a suitable electric field (which may be AC, DC, etc.) are known to those of ordinary skill in the art.

In some embodiments the fluid may be an electrical conductor. As used herein, a “conductor” is a material having a conductivity of at least about the conductivity of 18 megohm (MOhm) water. The liquid surrounding the fluid may have a conductivity less than that of the fluid. For example, the liquid may be an insulator, relative to the fluid, or at least a “leaky insulator,” i.e., the liquid is able to at least partially electrically insulate the fluid for at least a short period of time. The fluid may be substantially hydrophilic, and the liquid surrounding the fluid may be substantially hydrophobic.

Systems and methods may be provided for at least partially neutralizing an electric charge present on a fluidic droplet; for example, a fluidic droplet having an electric charge, as described herein. To at least partially neutralize the electric charge, the fluidic droplet may be passed through an electric field and/or brought near an electrode. Upon exiting of the fluidic droplet from the electric field (i.e., such that the electric field no longer has a strength able to substantially affect the fluidic droplet), and/or other elimination of the electric field, the fluidic droplet may become electrically neutralized, and/or have a reduced electric charge.

Nanoreactors may also be created from a fluid surrounded by a liquid within a channel by altering the channel dimensions in a manner that is able to induce the fluid to form individual droplets. For example, the channel may expand relative to the direction of flow, e.g., such that the fluid does not adhere to the channel walls and forms individual droplets instead, or the channel may narrow relative to the direction of flow such that the fluid is forced to coalesce into individual droplets. Internal obstructions may also be used to cause droplet formation to occur. Baffles, ridges, posts, or the like may be used to disrupt liquid flow in a manner that causes the fluid to coalesce into fluidic droplets.

In some embodiments, the channel dimensions may be altered with respect to time (for example, mechanically or electromechanically, pneumatically, etc.) in such a manner as to cause the formation of individual fluidic droplets to occur. For example, the channel may be mechanically contracted (“squeezed”) to cause droplet formation, or a fluid stream may be mechanically disrupted to cause droplet formation, for example, through the use of moving baffles, rotating blades, or the like.

Alternatively, individual fluidic droplets may be created and maintained in a system comprising three essentially mutually immiscible fluids (i.e., immiscible on a time scale of interest), where one fluid is a liquid carrier, and the second fluid and the third fluid alternate as individual fluidic droplets within the liquid carrier. In such a system, surfactants are not necessarily required to ensure separation of the fluidic droplets of the second and third fluids. Examples of systems involving three essentially mutually immiscible fluids include 1) a silicone oil, a mineral oil, and an aqueous solution; 2) a silicone oil, a fluorocarbon oil, and an aqueous solution; and 3) a hydrocarbon oil (e.g., hexadecane), a fluorocarbon oil (e.g. octadecafluorodecahydronaphthalene), and an aqueous solution.

Other examples of the production of droplets of fluid surrounded by a liquid are described in International Patent Application Serial No. PCT/US2004/010903, filed Apr. 9, 2004 by Link, et al. and International Patent Application Serial No. PCT/US03/20542, filed Jun. 30, 2003 by Stone, et al., published as WO 2004/002627 on Jan. 8, 2004, each incorporated herein by reference.

In some embodiments, the fluidic droplets may each be substantially the same shape and/or size. The shape and/or size can be determined, for example, by measuring the average diameter or other characteristic dimension of the droplets. Examples of suitable techniques include, but are not limited to, spectroscopy such as infrared, absorption, fluorescence, UV/visible, FTIR (“Fourier Transform Infrared Spectroscopy”), or Raman; gravimetric techniques; ellipsometry; piezoelectric measurements; immunoassays; electrochemical measurements; optical measurements such as optical density measurements; circular dichroism; light scattering measurements such as quasielectric light scattering; polarimetry; refractometry; or turbidity measurements.

Microfluidics

Microfluidic systems may be used for creating and handling nanoreactors. The use of microfluidic systems to create nanoreactors has a number of advantages. Advantages include the allowance of the formation of highly monodisperse nanoreactors, each of which functions as an almost identical, very small reactor. In addition, nanoreactors can have volumes ranging from femtoliters to nanoliters. Furthermore, compartmentalization in nanoreactors may prevent diffusion and dispersion due to parabolic flow. In some cases, use of a perfluorocarbon carrier fluid may prevent exchange of molecules between nanoreactors. In some cases, compounds in nanoreactors may not react or interact with the fabric of the microchannels as they are separated by a layer of carrier fluid; for example, inert perfluorocarbon. Another advantage of microfluidics is that nanoreactors may be created at up to and including 10,000 s⁻¹ and screened using optical methods at the same rate.

Nanoreactors may be fused or split using microfluidics. For example, aqueous microdroplets may be merged and split using microfluidics systems (Link, D. R., et al. (2004) Phys. Rev. Letts., 92:054503; Song, H., et al. (2003) Angew. Chem. Int. Ed. Engl., 42:767-772; WO 2007/089541). Nanoreactor fusion allows the mixing of reagents; for example, a nanoreactor containing a target may fuse with a nanoreactor containing the compound which could then initiate a reaction between target and compound. Nanoreactor splitting may allow single nanoreactors to be split into two or more smaller nanoreactors. For example a single nanoreactor containing a compound can be split into multiple nanoreactors which can then each be fused with different nanoreactors containing different targets. A single nanoreactor containing a target may also be split into multiple nanoreactors which may then each be fused with a different nanoreactor containing a different compound, or compounds at different concentrations.

Microfluidic systems and methods for splitting a fluidic droplet into two or more droplets have been described (see for example, U.S. Patent Application Publication No. US2007/0092914 A1). The fluidic droplet may be surrounded by a liquid, e.g., as previously described, and the fluid and the liquid are essentially immiscible in some cases. The two or more droplets created by splitting the original fluidic droplet may each be substantially the same shape and/or size, or the two or more droplets may have different shapes and/or sizes, depending on the conditions used to split the original fluidic droplet. In many cases, the conditions used to split the original fluidic droplet can be controlled in some fashion, for example, manually or automatically. In some cases, each droplet in a plurality or series of fluidic droplets may be independently controlled. For example, some droplets may be split into equal parts or unequal parts, while other droplets are not split.

A fluidic droplet may be split using an applied electric field. The electric field may be an AC field, a DC field, etc. The fluidic droplet, in this embodiment, may have a greater electrical conductivity than the surrounding liquid, and, in some cases, the fluidic droplet may be neutrally charged. The droplets produced from the original fluidic droplet are of approximately equal shape and/or size. In certain cases, in an applied electric field, electric charge may be urged to migrate from the interior of the fluidic droplet to the surface to be distributed thereon, which may thereby cancel the electric field experienced in the interior of the droplet. In some cases, the electric charge on the surface of the fluidic droplet may also experience a force due to the applied electric field, which causes charges having opposite polarities to migrate in opposite directions. The charge migration may, in some cases, cause the drop to be pulled apart into two separate fluidic droplets. The electric field applied to the fluidic droplets may be created, for example, using the techniques described above, such as with a reaction an electric field generator, etc.

Systems and methods for fusing or coalescing two or more fluidic droplets into one droplet are provided. For example, systems and methods to cause two or more droplets to fuse or coalesce into one droplet in cases where the two or more droplets ordinarily are unable to fuse or coalesce, for example, due to composition, surface tension, droplet size, the presence or absence of surfactants, etc. In some microfluidic systems, the surface tension of the droplets, relative to the size of the droplets, may also prevent fusion or coalescence of the droplets from occurring in some cases.

In some embodiments, two fluidic droplets may be given opposite electric charges (i.e., positive and negative charges, not necessarily of the same magnitude), which may increase the electrical interaction of the two droplets such that fusion or coalescence of the droplets can occur due to their opposite electric charges, e.g., using the techniques described herein. For example, an electric field may be applied to the droplets, the droplets may be passed through a capacitor, a chemical reaction may cause the droplets to become charged, etc.

Fluidic handling of nanoreactors has many advantages: nanoreactors can be split into two or more smaller nanoreactors allowing the reagents contained therein to be reacted with a series of different molecules in parallel or assayed in multiplicate; nanoreactors can be fused thereby allowing molecules to be diluted, mixed with other molecules, and reactions initiated, terminated or modulated at precisely defined times; reagents can be mixed very rapidly in nanoreactors using chaotic advection, allowing fast kinetic measurements and very high throughput; and reagents can be mixed in a combinatorial manner.

Creating and manipulating nanoreactors in microfluidic systems allows that stable streams of nanoreactors may be formed in microchannels and identified by their relative positions. If the reactions are accompanied by an optical signal (e.g. a change in fluorescence) a spatially-resolved optical image of the microfluidic network allows time resolved measurements of the reactions in each nanoreactors. Nanoreactors may be separated using a microfluidic flow sorter to allow recovery and further analysis or manipulation of the molecules they contain.

A variety of materials and methods may be used to form any of the above-described components of the microfluidic systems. In some embodiments, at least a portion of the fluidic system is formed of silicon by etching features in a silicon chip. Technologies for precise and efficient fabrication of various fluidic systems and devices of the invention from silicon are known. In some cases, various components of the systems and devices of the invention can be formed of a polymer, for example, an elastomeric polymer such as polydimethylsiloxane (“PDMS”), polytetrafluoroethylene (“PTFE” or Teflon®), or the like.

In some embodiments of the invention, sensors are provided that can sense and/or determine one or more characteristics of the fluidic droplets, and/or a characteristic of a portion of the fluidic system containing the fluidic droplet (e.g., the liquid surrounding the fluidic droplet) in such a manner as to allow the determination of one or more characteristics of the fluidic droplets. Characteristics determinable with respect to the droplet and usable in the invention can be identified by those of ordinary skill in the art. Non-limiting examples of such characteristics include fluorescence, spectroscopy (e.g., optical, infrared, ultraviolet, etc.), radioactivity, mass, volume, density, temperature, viscosity, pH, concentration of a substance, such as a biological substance (e.g., a protein, a nucleic acid, etc.), or the like.

In some embodiments of the invention, the microfluidic system includes a sensor. The sensor may be connected to a processor, which in turn, cause an operation to be performed on the fluidic droplet, for example, by sorting the droplet, adding or removing electric charge from the droplet, fusing the droplet with another droplet, splitting the droplet, causing mixing to occur within the droplet, etc., for example, as previously described. For example, in response to a sensor measurement of a fluidic droplet, a processor may cause the fluidic droplet to be split, merged with a second fluidic droplet, sorted etc.

One or more sensors and/or processors may be positioned to be in sensing communication with the fluidic droplet. “Sensing communication,” as used herein, means that the sensor may be positioned anywhere such that the fluidic droplet within the fluidic system (e.g., within a channel), and/or a portion of the fluidic system containing the fluidic droplet may be sensed and/or determined in some fashion. As an example, a laser may be directed towards the fluidic droplet and/or the liquid surrounding the fluidic droplet, and the fluorescence of the fluidic droplet and/or the surrounding liquid may be determined.

Non-limiting examples of sensors useful in the invention include optical or electromagnetically-based systems. For example, the sensor may be a fluorescence sensor (e.g., stimulated by a laser), a microscopy system (which may include a camera or other recording device), or the like. As another example, the sensor may be an electronic sensor, e.g., a sensor able to determine an electric field or other electrical characteristic. For example, the sensor may detect capacitance, inductance, etc., of a fluidic droplet and/or the portion of the fluidic system containing the fluidic droplet.

As used herein, a “processor” or a “microprocessor” is any component or device able to receive a signal from one or more sensors, store the signal, and/or direct one or more responses, for example, by using a mathematical formula or an electronic or computational circuit. The signal may be any suitable signal indicative of the environmental factor determined by the sensor, for example a pneumatic signal, an electronic signal, an optical signal, a mechanical signal, etc.

Screening/Sorting of Nanoreactors

Systems and methods for screening or sorting fluidic droplets in a liquid, and in some cases, at relatively high rates, are provided. For example, a characteristic of a droplet may be sensed and/or determined in some fashion, and then the droplet may be directed towards a particular region of the device, for example, for sorting or screening purposes. In some cases a characteristic of a fluidic droplet may be sensed and/or determined in some fashion, for example, fluorescence of the fluidic droplet may be determined, and, in response, an electric field may be applied or removed from the fluidic droplet to direct the fluidic droplet to a particular region (e.g. a channel). A fluidic droplet may be directed by creating an electric field on the droplet and steering the droplet using an applied electric field; for example an AC field, a DC field, etc. In some cases, a fluidic droplet may be sorted and steered by inducing a dipole in the fluidic droplet, which may be initially charged or uncharged, and sorting or steering the droplet using an applied electric field.

Fluidic droplet may be screened or sorted within a fluidic system of the invention by altering the flow of the liquid containing the droplets. For example, a fluidic droplet may be steered or sorted by directing the liquid surrounding the fluidic droplet into a first channel, a second channel, etc.

Pressure within a fluidic system, for example, within different channels or within different portions of a channel, may be controlled to direct the flow of fluidic droplets. For example, a droplet may be directed toward a channel junction including multiple options for further direction of flow (e.g., directed toward a branch, or fork, in a channel defining optional downstream flow channels). Pressure within one or more of the optional downstream flow channels can be controlled to direct the droplet selectively into one of the channels, and changes in pressure can be effected on the order of the time required for successive droplets to reach the junction, such that the downstream flow path of each successive droplet can be independently controlled. In one variation, the expansion and/or contraction of liquid reservoirs may be used to steer or sort a fluidic droplet into a channel, e.g., by causing directed movement of the liquid containing the fluidic droplet. The liquid reservoirs may be positioned such that, when activated, the movement of liquid caused by the activated reservoirs causes the liquid to flow in a preferred direction, carrying the fluidic droplet in that preferred direction. In some cases, the expansion and/or contraction of the liquid reservoir may be combined with other flow-controlling devices and methods.

Fluidic droplets may be sorted into more than two channels. In some cases, droplets desired droplets may be segregated and remaining droplets may be returned to a starting pool of droplets for further use.

In some embodiments, the nanoreactors or microbeads are analyzed and, optionally, sorted by flow cytometry. Many formats of nanoreactor can be analyzed and, optionally, sorted directly using flow cytometry.

Flow analysis and flow sorting (Fu, A. Y., et al. (2002) Anal Chem, 74:2451-2457) using microfluidic devices may be used in screening and sorting of nanoreactors. A variety of optical properties can be used for analysis and to trigger sorting, including light scattering (Kerker, M. (1983) Cytometry 4(1):1-10) and fluorescence polarization (Rolland, J. M. et al. (1985) J Immunol Methods 76(1):1-10). In some cases, the difference in optical properties of the nanoreactors or microbeads will be a difference in fluorescence and the nanoreactors may be sorted using a microfluidic or conventional fluorescence activated cell sorter (Norman, A. (1980) Med Phys 7(6):609-15; Mackenzie, N. M. and Pinder, A. C. (1986) Dev Biol Stand. 64:181-93), or similar device. Advantages of flow cytometry include (1) fluorescence activated cell sorting equipment from established manufacturers (e.g. Becton-Dickinson, Coulter, Cytomation) allows the analysis and sorting at up to 100,000 nanoreactors or microbeads; (2) the fluorescence signal from each nanoreactor or microbead corresponds tightly to the number of fluorescent molecules present; (3) the wide dynamic range of the fluorescence detectors (typically 4 log units) allows easy setting of the stringency of the sorting procedure, thus allowing the recovery of the optimal number of nanoreactors from the starting pool; (4) fluorescence-activated cell sorting equipment can perform simultaneous excitation and detection at multiple wavelengths (Shapiro, H. M. (1995). Practical Flow Cytometry, 3 ed, New York, Wiley-Liss) allowing positive and negative selections to be performed simultaneously. If the nanoreactors or microbeads are optically tagged, flow cytometry may also be used to identify the compound or compounds in the nanoreactors. Optical tagging can also be used to identify the concentration of the compound in the nanoreactor or the number of compound molecules coated on a microbead. Furthermore, optical tagging can be used to identify the target in a nanoreactor. This analysis can be performed simultaneously with measuring activity, after sorting of nanoreactors containing microbeads, or after sorting of the microbeads.

### EXAMPLES

The following Example is provided to illustrate but not limit the invention.

### Example 1

### Quantification of Cytokine mRNA in Peripheral Blood Mononuclear Cells using Nanoreactor Technology

A branched DNA (bDNA) signal amplification assay (Shen, L P et al. 1998 J. Immunological Methods 215:123-134) is used to quantify cytokine mRNA in peripheral blood mononuclear cells (PBMCs).

Blood is collected from an individual into EDTA anticoagulant tubes and processed within two hours of collection. PBMCs are isolated using Leucoprep tubes containing sodium citrate (Becton Dickinson) or by centrifugation over sterile 60% Percoll gradients. Cell numbers are determined by hemocytometer. Cell pellets are stored at −80° C.

A sample containing mRNA is nano-aliquoted into nanoreactors and collected as described above. Samples may be in the form of cells, such as PBMC, lysed cells or isolated mRNA.

A second set of bDNA assay nanoreactors containing labeled extenders, capture extenders and capture probes is prepared as described above. Label extenders are designed to have a portion complementary to the target mRNA and a second segment complementary to a bDNA amplifier. Capture extenders are designed to have a portion complementary to the target mRNA and a second segment complementary to a capture probe. Capture probes are designed to be complementary to capture extenders and are bound to a solid phase. The solid phase is coded for selection and detection.

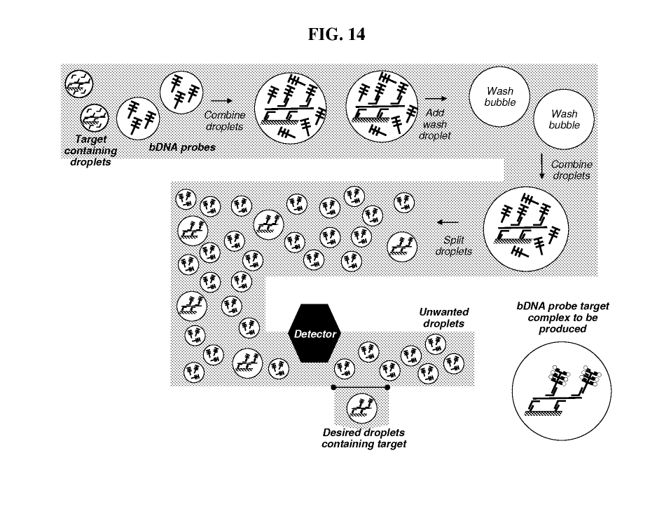

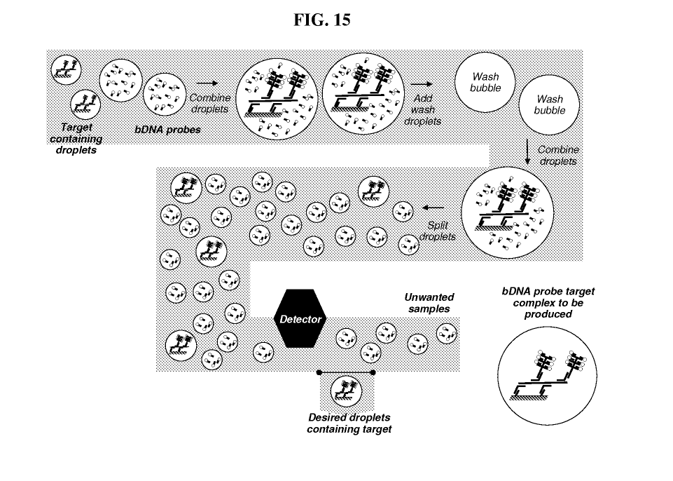

The reaction is initiated by the addition of proteinase K and SDS to lyse cells if needed. Proteinase K and SDS are included in either the sample nanoreactor, the assay component nanoreactor or in a third nanoreactor. Nanoreactors are combined and incubated at 53° C. or 63° C. overnight (FIG. 13). Nanoreactors are cooled to room temperature for 10 min and washed with nanoreactors containing Wash A (0.1× Standard Sodium Citrate [SSC; 1×SSC is 0.15 M sodium chloride, 0.015 sodium citrate], 0.1% sodium dodecyl sulfate [SDS]) as described above to reduce excess reaction components and sample debris. Multiple wash steps may be performed. Washed nanoreactors are then combined with nanoreactors containing bDNA amplifiers in amplifier diluent which hybridize to the label extender (FIG. 14). (Amplifier diluent is prepared by mixing 50% horse serum, 1.3% SDS, 6 mM Tris-HCl, pH 8.0, 5×SSC and 0.5 mg/ml proteinase K and incubating at 65 C for 2 hr followed by adding 1 mM phenylmethylsulfonyl fluoride to inactivate the proteinase K and 0.05% each of sodium azide and Proclin 300). Combined nanoreactors are incubated at 53° C. for 30 min and then cooled to room temperature for 10 min. Nanoreactors are then combined with wash nanoreactors as above. Desired washed samples are then separated based on the coded solid support portion of the capture probe. Collected nanoreactors are then combined with nanoreactors containing a labeled probe that is complementary to multiple copies of an oligonucleotide complement within the bDNA amplifier (FIG. 15). The combined nanoreactors are washed with a wash nanoreactor as described above. Nanoreactors containing the coded solid phase are analyzed using a suitable detector system such as the PLS detector system. The amount of labeled probe bound to the solid phase is proportional to the mRNA in the sample.

### Example 2

### Quantification of Angiogenin (ANG) in a Blood Serum Sample using Nanoreactor Technology

A heterogeneous sandwich immunoassay in nanoreactors is used to quantify the concentration of ANG in a serum sample. In this example the capture antibody is conjugated to a bead, the presence of which can be measured for example optically. The detection antibody is labeled to facilitate the assay read out.

A standard serum sample is nano-aliquoted into nanoreactors and collected as described above. For this particular assay prior to forming sample droplets the serum sample should be diluted to an appropriate level to produce a concentration dependent signal based on a standard curve.

Reagents droplets are prepared from a solution of the capture antibody attached to a particle, in this case a bead, and the detection antibody in Reagent Diluent 1 (RD1; 1%BSA/PBS, pH 7.2-7.4, 0.2 uM filtered). The particles are coded for selection and detection. In this example the capture antibody conjugated beads are prepared by first washing MyOne beads 1× with an appropriate buffer, such as one that does not contain a primary or secondary amine. The beads are then suspended in the appropriate buffer at pH 7, 1-ethyl-3-(3-dimethylaminopropyl)carbodiimide hydrochloride (EDC) and sulfo-N-hydroxy succinimide (sulfo-NHS) are added the mixture incubated. The beads are then diluted with phosphate buffered saline (PBS), washed once with PBS, resuspended in PBS, the anti-ANG antibody is added and the mixture incubated. The beads are then isolated, washed with PBS 1× and a buffer containing a primary amine such as Tris or glycine is added. After incubation the beads are washed with PBS. Prior to aliquoting into droplets the beads should be washed with RD1 1× and incubated with in RD1 for example for 1 hour.

The sample droplets and the reagent droplets are combined and incubated at ambient temperature. The droplets containing the sandwich complex are then washed with nanoreactors containing Wash Buffer A (WBA; 0.05% Tween 20/PBS pH7.2-7.4, 0.2 uM filtered) as described above to reduce excess reaction components and sample debris. Multiple wash steps may be performed. Following washing, the droplets containing the sandwich complex (recognized by way of the particle conjugated to the capture antibody) are then combined with droplets containing streptavidin-horseradish peroxidase (SA-HRP) in RD1 buffer. The combined droplets are incubated and then washed with wash droplets containing WBA as described above. Multiple wash steps may be performed. The particle containing droplets are then washed with PBS. Following washing the droplets containing the antibody-conjugated particles are combined with droplets containing HRP substrates that produce fluorescent products. After an incubation period the fluorescent signals in the droplets are measured and the amount of ANG in the original sample determined by comparing the signal obtained with a standard curve. The amount of HRP signal is proportional to the concentration of the ANG in the sample.

### Example 3

### Quantification of Low Abundance Analytes using Nanoreactors

This approach is specifically directed toward analyzing low abundance analytes in a sample. The following example uses the same basic immunoassay as described in Example 2 as an example assay however the approach could be adapted for other assay platforms used in nanoreactors. In this example the capture antibody is conjugated to a bead, the presence of which can be measured, for example optically. The detection antibody is labeled to facilitate the assay read out.

Sample and reagent droplets are prepared as described in Example 2. In the case of the sample droplets and very low abundance analytes, for example Protein X, there is a point dependent of droplet volume and analyte concentration in the bulk sample where some sample droplets will not contain Protein X and those that do contain Protein X have only have a low number of Protein X. At more of an extreme case sample droplet may either only contain one analyte or none at all. A sample could be diluted to make sure this was the case for higher concentration analytes. The single molecule per droplet with multiple sample droplets not containing the analyte would be the case for example if Protein X is at 10 aM concentration in the bulk sample and the sample is divided into a plurality of 20 um diameter droplets each ˜4.2 pL where Protein X would then be at 0.39 pM in droplet containing a single molecule of Protein X and 0 M in droplets not containing Protein X. An assay used in any particular droplet would then only need a lower limit of detection that is sufficient for the single molecule concentration in a single droplet.

For this example, Protein X at 10 aM in the bulk serum, 120,000 droplets at 20 um diameter would generate approximately 3 droplets each containing one molecule of Protein X and the rest not containing Protein X. Those droplets would then be processed as described in Example 2 using an immunoassay specific for Protein X and a detection method that could generate a signal for a single Protein X molecule, for example using a poly SA-HRP label that is capable of generating >10× the signal of a standard SA-HRP conjugate. The fluorescent signals in the droplets are measured and the amount of Protein X in the original sample is determined by calculating how many molecules were in the original sample (120,000 droplets@4.2 pL each or 0.5 uL) based on how many droplets have signal. In this case there would be three droplets with signal so 3 molecules in 0.5 uL volume equals 10 aM in the original bulk sample. Of course the accuracy of this method increases with the more analyte containing droplets detected (i.e. more droplets that produce a signal).

### Example 4

### Tracking which Sample Droplets are Combined with Which Reagent Droplets

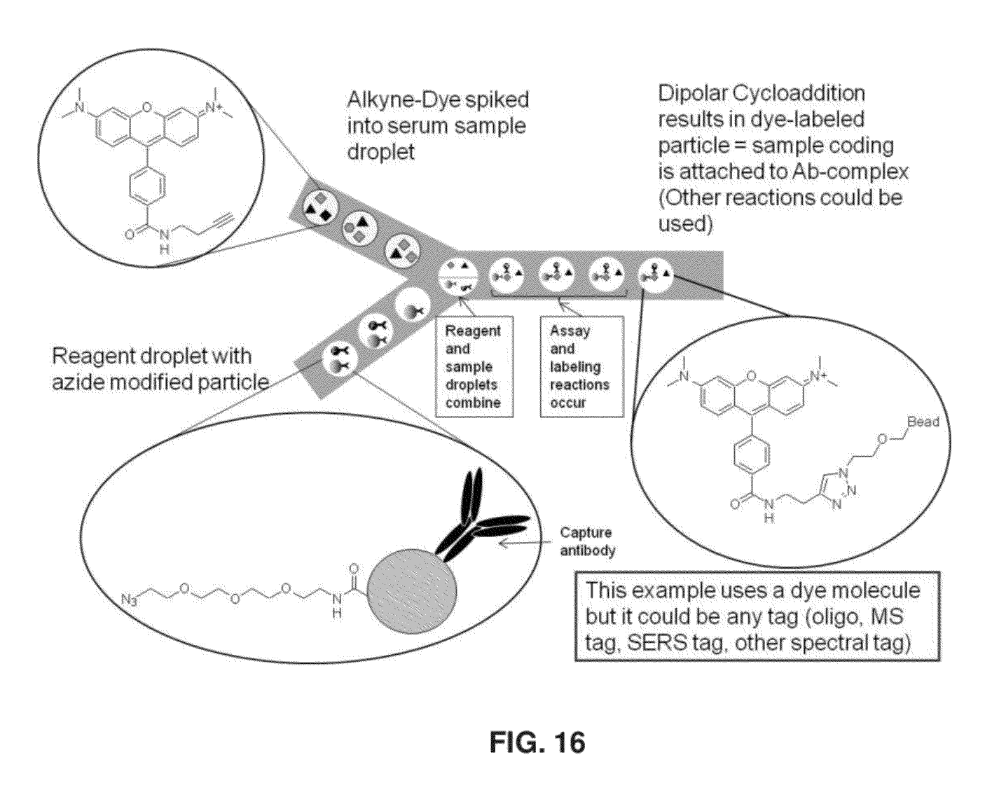

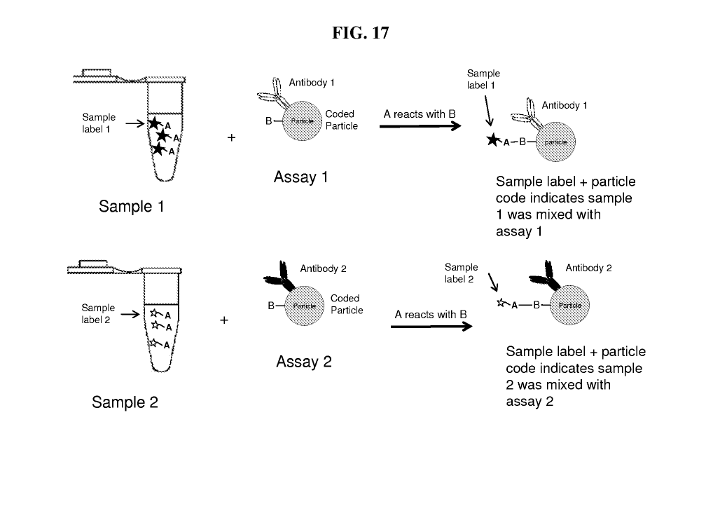

The following example uses the same basic immunoassay as described in Example 2 as an example assay however the approach could be adapted for other assay platforms used in nanoreactors. In this example the sample is spiked with a reporter such as a fluorescent dye that also contains a first reactive group an alkyne in this case. See FIGS. 16 and 17. Multiple different dyes are used to label different samples such that each sample has a unique dye signature all with a first reactant. The capture antibody is conjugated to a bead, the presence of which can be measured and identified, for example optically. Multiple different reagents corresponding to different assays have differently optically labeled beads such that each identifies the reagents in the droplet. The beads have also been modified with a second reactive group; an azide in this case. This can be done for example by adding the appropriate amino-azide compound to the EDAC activated carboxylate beads either with the capture antibody or after an initial incubation with the antibody as described in Example 2. The detection antibodies are labeled to facilitate the assay read out.

Reagent and Sample droplets are prepared and combined as described in Example 2. In this case multiple different sample droplets are combined with multiple different reagent droplets. When the droplets are combined in addition to the desired sandwich immunoassay complex forming in the presence of analyte, the two complementary reactive functional groups, the first react from the sample and the second reactant on the bead, also react. In this case the reaction is a [3+2] cycloaddition to form a triazole. This reaction may or may not be catalyzed by an additional agent such as copper ions. When this reaction occurs the dye that is a specific label for the sample identity is transferred to the bead which is specific for the reagents and assay resulting in an additional label on the bead. The identification of both the bead label and the newly conjugated dye label allows for the tracking of the combination of this particular sample droplet with this particular reagent droplet.

The droplets are further processed as outlined in Example 2. Three measurements are made after adding the assay reagents (the substrate to generate the HRP signal): 1) the HRP signal indicating the presence of the analyte protein X, the dye label from the sample that is now conjugated the particle from the reagent droplet and finally the signature of the particle that encodes which reagents were in the original reagent droplet.

### Example 5

### Heterogeneous Assays: Washing using Magnetophoresis

The following example uses the same basic immunoassay as described in Example 2 with the difference in the method of washing. This washing method utilizes magnetic forces to divert magnetic beads within the nanoreactors to provide extremely efficient washing. The MyOne beads described in Example 2 are magnetic but other magnetic beads could be used. In addition, beads can either be coded or not coded for downstream identification of the assay performed.

Following the combination of reagent and sample beads and the subsequent incubation beads are flowed into the microfluidic magnetophoretic separation device. The magnetic field create by the device diverts the magnetic beads from the sample stream to a separate fluidic stream separated by laminar flow. The new stream in one example is comprised of the wash buffer. By controlling the flows of both solutions either the entire immunoassay droplet can be diverted into the new stream or the magnetic bead can be diverted out from the main droplet resulting in a much smaller droplet containing the magnetic bead being diverted into the new fluidic stream. New droplets could be formed from the new stream and the process repeated for additional washing steps.

An alternative approach to the same basic example would use a second stream that maintains the emulsion (i.e. it is oil if the droplets are aqueous). If the entire droplet is deflected into this new oil stream then this approach would be used in combination with the washing approach outlined in Example 2 where wash droplets are combined with the assay droplets. The magnetic sorting would be used to isolate the post-wash droplets containing the magnetic particles. If the magnetophoretic device is used to pull the magnetic particles out of the main droplet, in the process forming a smaller droplet then the new smaller droplets would be combined with a wash droplet and the process repeated until sufficient washing is achieved.

After washing, the rest of the immunoassay is completed as outlined in Example 2 with all subsequent wash steps being achieved with one version of methods described here for using the magnetophoretic device. The assay is read our as described for Example 2.

Antibody approach could be adapted for other assay platforms used in nanoreactors. In this example the sample is spiked with a reporter such as a fluorescent dye that also contains a first reactive group an alkyne in this case. Multiple different dyes are used to label different samples such that each sample has a unique dye signature all with a first reactant. The capture antibody is conjugated to a bead, the presence of which can be measured and

Although the foregoing invention has been described in some detail by way of illustration and example for purposes of clarity of understanding, the descriptions and examples should not be construed as limiting the scope of the invention.

What is claimed is:

1. A method of washing a nanoreactor containing a particle in a microfluidic system, comprising the steps of:
a) fusing a first nanoreactor containing a particle with a second nanoreactor containing a washing solution to form a combined nanoreactor, wherein the diameter of the second nanoreactor is at least about two fold the diameter of the first nanoreactor; and wherein a molecule within the first nanoreactor is diluted in the combined nanoreactor;
b) splitting the combined nanoreactor into a plurality of nanoreactors; and
c) separating the nanoreactor containing the particle from the plurality of nanoreactors formed in step b) in a microfluidic system.

2. The method of claim 1, wherein the diameter of the second nanoreactor is at least about five fold of the diameter of the first nanoreactor.

3. The method of claim 1, wherein the diameter of the second nanoreactor is at least about ten fold of the diameter of the first nanoreactor.

4. The method of claim 1, wherein the particle comprises a reporter

5. The method of claim 4, wherein the reporter is a dye coded bead or a nano-bar code.

6. The method of claim 1, wherein the first nanoreactor has a cross-sectional dimension of less than about 100 microns.

7. The method of claim 1, wherein the first nanoreactor has a cross-sectional dimension of less than about 30 microns.

8. The method of claim 1, wherein the first nanoreactor has a cross-sectional dimension of less than about 10 microns.

9. The method of claim 1, wherein the first nanoreactor has a cross-sectional dimension of less than about 3 microns.

10. The method of claim 1, wherein the method is used in a washing step of a heterogeneous assay.

11. The method of claim 1, wherein the plurality of nanoreactors which do not contain the particle are returned to a starting pool for further analysis.

12. A method for tracking a sample in a nanoreactor comprising the steps of a) fusing a sample nanoreactor comprising a sample and a reporter with a reagent nanoreactor comprising a particle and a reagent, wherein the reporter comprises a first reactive group, and a second reactive group and a reagent are associated with the particle; wherein the first reactive group reacts with the second reactive group so that the reporter is linked to the particle; and b) tracking the sample nanoreactor that has reacted with the reagent nanoreactor by tracking the nanoreactor containing the reporter.

13. The method of claim 12, wherein the reporter is covalently linked to the particle in step a).

14. The method of claim 12, wherein the reporter is not covalently linked to the particle in the step a).

15. The method of claim 12, wherein the reporter is selected from the group consisting of a dye, a fluorescent agent, an ultraviolet agent, a chemiluminescent agent, a chromophore, a radio-label, a mass spectrometry tag molecule, and a resonance raman tag molecule.

16. A method of measuring concentration of an analyte in a sample, said method comprising:
a) compartmentalizing a sample into a plurality of nanoreactors, wherein at least about 80% of the nanoreactors contain no more than a single analyte molecule; and
b) detecting the nanoreactor containing at least an analyte molecule; wherein the number of nanoreactors containing analyte molecules indicates the concentration of the analyte in the sample.

17. The method of claim 16, wherein at least about 90% of the nanoreactors contain no more than a single analyte molecule.

18. The method of claim 16, wherein at least about 95% of the nanoreactors contain no more than a single analyte molecule.

19. The method of claim 16, wherein greater than 95% of the nanoreactors contain no more than a single analyte molecule.

20. The method of claim 16, wherein the analyte containing nanoreactors are detected by labeling the analyte with a reporter.

21. The method of claim 16, wherein the concentration of the analyte in the sample is about 5 aM to about 500 fM.

22. The method of claim 16, wherein the analyte is selected from the group consisting of a protein, a peptide, an oligonucleotide, a metabolite, a carbohydrate, a lipid, a ligand, a receptor, and a small molecule.

23. The method of claim 16, wherein the sample is a clinical sample selected from the group consisting of blood, plasma, serum, saliva, urine, and spinal fluid.

24. The method of claim 16, wherein the nanoreactors have a cross-sectional dimension of less than about 100 microns.

25. The method of claim 16, wherein the nanoreactors have a cross-sectional dimension of less than about 30 microns.

26. The method of claim 16, wherein the nanoreactors have a cross-sectional dimension of less than about 10 microns.
